(19) United States
(12) Patent Application Publication
(10) Pub. No.: US 2017/0231739 A1
(43) Pub. Date: Aug. 17, 2017

(54) HYBRID SURGICAL MESH

(71) Applicant: Vadim MOROZOV, Towson, MD (US)

(72) Inventor: Vadim MOROZOV, Towson, MD (US)

(21) Appl. No.: 15/502,216

(22) PCT Filed: Aug. 7, 2015
(86) PCT No.: PCT/US15/44299

§ 371 Date: Feb. 7, 2017

Related U.S. Application Data
(60) Provisional application No. 62/034,349, filed on Aug. 7, 2014.

(51) Int. Cl. A61F 2/00

(52) U.S. Cl. CPC A61F 2/0045; A61F 2250/003; A61F 2210/0004

(57) ABSTRACT

Disclosed is a hybrid surgical mesh that is partially absorbable or biodegradable. The hybrid surgical mesh described herein generally comprises a central absorbable or biodegradable portion surrounded on at least its sides by a non-absorbable portion which forms a support structure for supporting a prolapsed region. While the absorbable or biodegradable portion provides support to the prolapsed region immediately following the surgical procedure to correct such prolapse, over time the central absorbable or biodegradable portion dissolves so as to leave only the non-absorbable interrupted hammock-like support structure. Such configuration provides the initial structural support required after the surgical procedure necessary to ensure healing and strengthening of the prolapsed region, while significantly reducing the risk of healing abnormalities or complications resulting in the central portion of the graft.

### CROSS-REFERENCE TO RELATED APPLICATION

This application is based upon and claims the benefit of copending U.S. Provisional Patent Application Ser. No. 62/034,249 entitled “Hybrid Surgical Mesh,” filed with the U.S. Patent and Trademark Office on Aug. 7, 2014 by the inventor herein, the specification of which is incorporated herein by reference in its entirety.

### FIELD OF THE INVENTION

The present invention relates generally to implantable surgical meshes, and more particularly to hybrid surgical meshes that are partially bio-absorbable.

### BACKGROUND OF THE INVENTION

Surgical meshes are used in a variety of surgical procedures. One particular application of surgical meshes is in the repair of pelvic organ prolapse (POP) in gynecologic surgery. POP occurs when tissues supporting the female pelvic organs become weak or stretched, and may result from a variety of conditions including pregnancy and childbirth, obesity, pelvic organ cancers, surgical removal of the uterus, and the like. The condition allows for organs, such as the bladder, uterus, or rectum, to bulge into the vagina and, in some extreme cases, to bulge past the vaginal opening. According to the United States Food and Drug Administration, approximately thirty five percent of women will experience POP in their lifetime, with two percent experiencing symptoms from the condition.

Transvaginal meshes have previously been provided and have become the preferred method of treating POP. The standard transvaginal surgical graft configuration consists of a generally rectangular solid sheet of macroporous, non-absorbable monofilament polypropylene mesh with four arms that extend laterally outward, which graft is permanently implanted in the patient's vagina. Each arm of the graft is attached to the pelvic side wall and/or anatomical support structures. After implantation, the graft serves as a floor of support for the bladder and other organs, adding support to the anterior-lateral vaginal walls.

Unfortunately, healing abnormalities and other complications arise in the use of such surgical meshes, and from 2008 through 2010, there were over 2800 reports of complications associated with surgical mesh devices. The most frequent complication was mesh erosion through the vagina. Such complications lead to pain (including during sexual intercourse), infection, bleeding, organ perforation, and urinary problems. Many of those complications require additional medical or surgical treatment.

In light of the foregoing limitations of previously known devices and methods, there remains a need in the art for a surgical graft that will provide the necessary support but that will not be subject to mesh erosion (i.e., loss of tissue surrounding the mesh).

### SUMMARY OF THE INVENTION

Disclosed herein is a hybrid surgical mesh that is partially absorbable or biodegradable. While the embodiments described herein are generally described to repair POP, different embodiments may be used to repair other complications such as rectocele prolapses or hernias.

The hybrid surgical mesh described herein generally comprises a central absorbable or biodegradable portion surrounded on at least its sides by a non-absorbable portion. The non-absorbable portion forms a hammock-like support structure for attaching the hybrid mesh within the patient's vagina and lateral pelvic walls in order to support a prolapsed region. While the absorbable or biodegradable portion is sufficiently strong to provide support to the prolapsed region immediately following the surgical procedure to correct such prolapse, over time the central absorbable or biodegradable portion dissolves so as to leave only the non-absorbable portion. Such configuration provides the initial structural support required after the surgical procedure necessary to ensure healing and strengthening of the prolapsed region, while significantly reducing the risk of healing abnormalities or complications resulting in the central portion of the graft.

In accordance with certain aspects of an embodiment of the invention, an implantable graft is provided comprising a hammock-like support structure configured for attachment to a patient's tissue formed of a non-absorbable mesh, and a biodegradable portion positioned at an interior portion of the non-absorbable mesh.

In accordance with further aspects of an embodiment of the invention, a mesh is provided for use in an implantable graft, comprising a biodegradable portion, and a non-absorbable mesh attached to at least a portion of an outer perimeter edge of the biodegradable portion.

### BRIEF DESCRIPTION OF THE DRAWINGS

The numerous advantages of the present invention may be better understood by those skilled in the art by reference to the accompanying figures in which:

FIG. 1 is a perspective view of a hybrid surgical mesh in accordance with certain aspects of an embodiment of the invention.

FIG. 2 is a perspective view of a hybrid surgical mesh in accordance with further aspects of an embodiment of the invention.

FIG. 3 is a perspective view of a hybrid surgical mesh in accordance with still further aspects of an embodiment of the invention.

FIG. 4 is a photograph of a hybrid mesh formed for testing purposes and in accordance with certain aspects of an embodiment of the invention.

FIG. 5 is a close-up photograph of another hybrid mesh formed for testing purposes and in accordance with certain aspects of an embodiment of the invention.

### DETAILED DESCRIPTION OF THE PREFERRED EMBODIMENTS

The invention summarized above may be better understood by referring to the following description, claims, and accompanying drawings. This description of an embodiment, set out below to enable one to practice an implementation of the invention, is not intended to limit the preferred embodiment, but to serve as a particular example thereof. Those skilled in the art should appreciate that they may readily use the conception and specific embodiments disclosed as a basis for modifying or designing other methods and systems for carrying out the same purposes of the present invention. Those skilled in the art should also realize that such equivalent assemblies do not depart from the spirit and scope of the invention in its broadest form. Further, it should be understood that the figures are not drawn to scale and in some instances details that are not necessary for the understanding of the present invention are omitted such as common methods of manufacturing.

Hybrid surgical meshes that are partially bio-absorbable or bio-degradable and partially non-degradable are described herein. While, for purposes of explanation, the hybrid surgical mesh set forth herein is described largely in the context of a surgical mesh used to repair POP, the invention is not limited to this context. Rather, the hybrid surgical meshes described herein may likewise be shaped for other medical implications such as, for example, rectal prolapse, enterocele, hernias, or such other medical conditions as may occur to those skilled in the art.

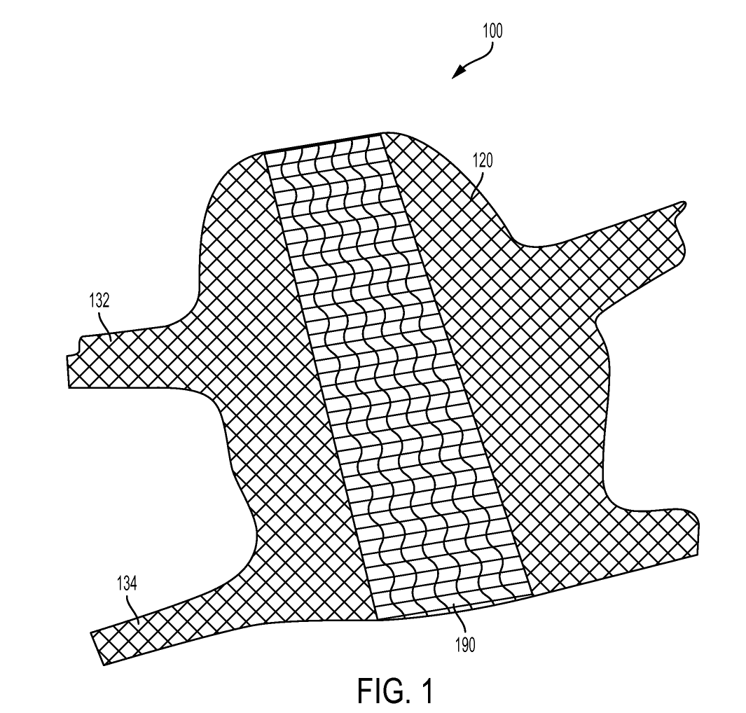

As explained above, previously known surgical grafts have carried the disadvantage of frequent healing abnormalities and complications, particularly including erosion. The inventor herein has discovered, however, that the center area 190 of a graft 100 formed in accordance with at least certain aspects of the instant invention (as shown in FIG. 1) is only temporarily needed after surgical insertion to support the continuity of the prolapsed area. Thus, by forming at least a portion of the center area 190 completely of a degradable material, a hybrid surgical graft 100 formed in accordance with the instant specification may avoid future complications associated with central portion graft erosion.

In accordance with certain aspects of an embodiment of the invention, FIG. 1 depicts a hybrid surgical graft 100 including a biodegradable central portion 190, with non-absorbable lateral portions 120 on each side of biodegradable central portion 190. Preferably, one or more arms 132 and 134 extend outward from non-absorbable lateral portions 120 to attach the hybrid surgical graft 100 to the pelvic sidewall. Hybrid surgical graft 100 also generally has an extended portion at the anterior end, which can be cut to fit the length of the patient's vagina. In an exemplary configuration, hybrid surgical graft 100 may be generally about 5 centimeters wide by 10 centimeters long, and arms 132 and 134 may be about 4 centimeters in length. Biodegradable central portion 190 may be formed from a biodegradable prosthetic material, a xenograft, an allograft, or homograft material. Likewise, non-absorbable lateral portions 120 may be formed from a prosthetic material such as polypropylene. Non-absorbable lateral portions 120 may contain pores throughout the sheet to allow for tissue integration. Biodegradable central portion 190 is configured to maintain its structural integrity until the patient's tissue has had sufficient time to fixate onto non-absorbable lateral portions 120 through fibrosis, as well as cause the fibrosis of the biodegradable central portion 190. In certain configurations, biodegradable central portion 190 is designed to lose its structural integrity between one to twelve months. In other configurations, biodegradable central portion 190 is designed to degrade in about one, two, three, four, five, six, seven, eight, nine, ten, eleven, or twelve months. In light of the teachings herein, a person having ordinary skill in the art would be able to select a material having the desired properties. Structural integrity, as defined herein, is the point when the absorbable or biodegradable central portion 190 of the hybrid surgical graft 100 is no longer supporting the previously prolapsed area. Once biodegradable central portion 190 is completely degraded or absorbed, hybrid surgical graft 100 remains permanently in the patient as two separate pieces.

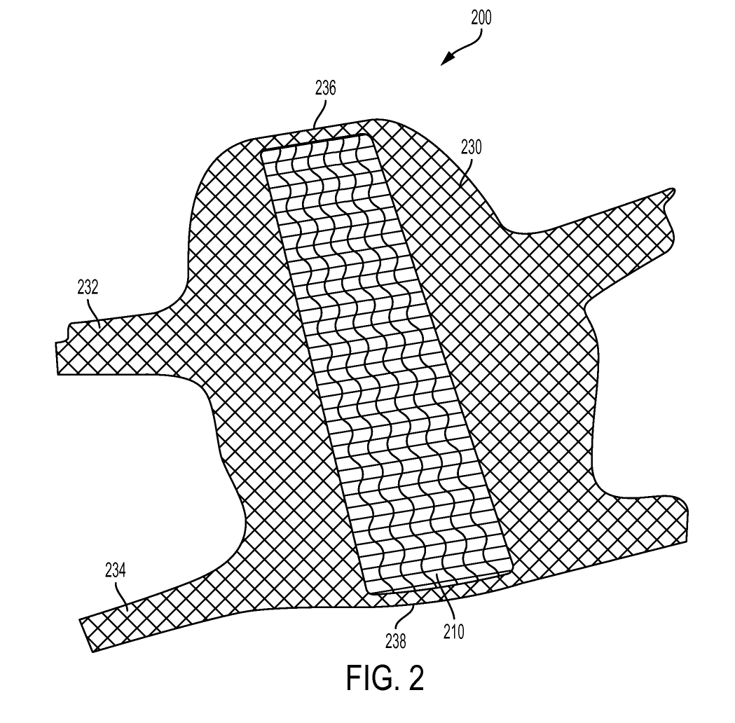

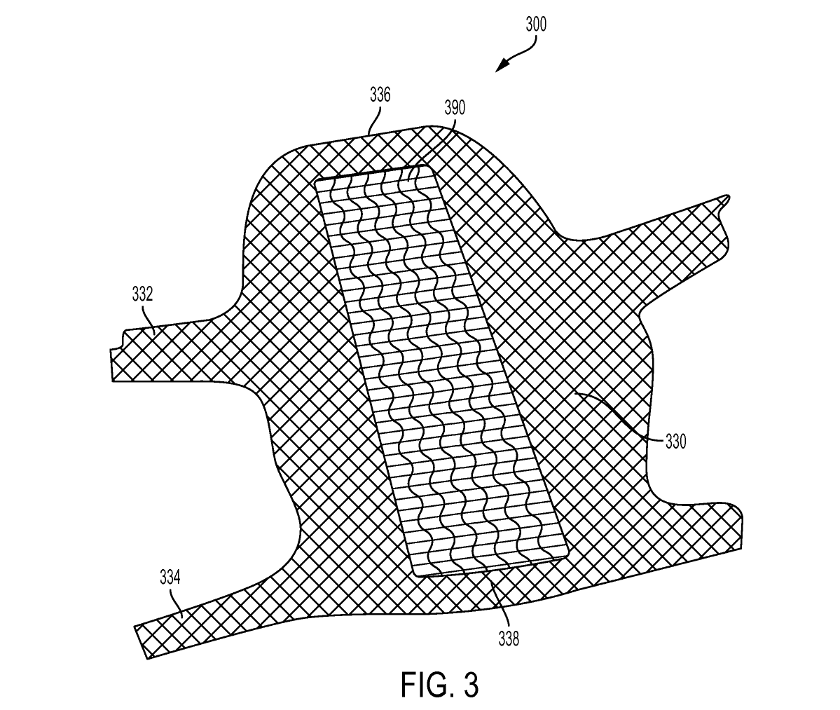

FIGS. 2 and 3 show additional configurations employing various aspects of the invention. Each of the configurations shown in FIGS. 2 and 3 include permanent, non-absorbable lateral portions on either side of the biodegradable or absorbable central portion.

With particular regard to FIG. 2, a hybrid surgical graft 200 in accordance with certain aspects of the invention may be provided with biodegradable central portion 210 attached along its long side edges to non-absorbable lateral side portions 230 (with preferably one or more arms 232 and 234 extending outward from non-absorbable lateral portions 230 to attach the hybrid surgical graft 200 to the pelvic sidewall), along its top edge to a permanent, non-absorbable anterior bridge 236, and along its bottom edge to permanent, non-absorbable posterior bridge 238, such that the entire perimeter of biodegradable central portion 210 is in contact with a section of non-absorbable material. In this configuration, following fibrosis and dissolution of central degradable portion 210, an annular ring of permanent, non-absorbable material will remain to provide continued support to the patient's vaginal walls, but with minimized risk of healing abnormalities or complications in the region that was initially covered by central bio degradable portion 210.

Next, and with particular regard to FIG. 3, hybrid surgical graft 300 may be provided with biodegradable central portion 390 attached along its long side edges to non-absorbable lateral side portions 330 (with preferably one or more arms 332 and 334 extending outward from non-absorbable lateral portions 330 to attach the hybrid surgical graft 300 to the pelvic sidewall), along its top edge to a permanent, non-absorbable anterior bridge 336 of increased width, and along its bottom edge to permanent, non-absorbable posterior bridge 338, such that the entire perimeter of biodegradable central portion 390 is again in contact with a section of non-absorbable material. In this configuration, following fibrosis and dissolution of central degradable portion 390, a reinforced annular ring of permanent, non-absorbable material will remain to provide continued support to the patient's vaginal walls, but with minimized risk of healing abnormalities or complications in the region that was initially covered by central bio degradable portion 390.

Anterior and posterior non-absorbable bridges 236 and 238 of FIG. 2 may have a width of approximately 1 mm, while anterior and posterior non-absorbable bridges 336 and 338 of FIG. 3 may have a width of approximately 2 mm. However, such dimensions are exemplary, and those of ordinary skill in the art will recognize that the width of such bridges may be increased or decreased to provide greater or less reinforcement to the resulting annular ring of permanent, non-absorbable material as a particular patient's condition may warrant, all without departing from the spirit and scope of the invention. However, in each of these configurations, the widths of the lateral and bridge portions are less than the length dimension along the major axis of the absorbable or degradable central portion.

The overall size of the hybrid surgical graft and the arms, including the dimensions and optimal pore size, would be readily selected and configured to meet the particular application in which the hybrid surgical graft configured as described herein is to be used by persons having ordinary skill in the art. In a particularly preferred configuration, the width of the absorbable or bio-degradable central portion is between approximately 0.5 centimeters and 5 centimeters. In certain configurations, the width of the central portion is about one, two, three, four, or five centimeters. The length of the central biodegradable portion (measured along the major axis of the absorbable or bio-degradable central portion) will depend on the size of the hybrid surgical graft, but is generally between about 1 centimeter and 10 centimeters. However, in some configurations the length of the central biodegradable portion can be about three, four, five, six, seven, eight, nine, or ten centimeters.

In a particularly preferred configuration of the hybrid surgical graft configured as described herein, permanent non-absorbable lateral portions and bridge portions are formed of a polypropylene non-absorbable mesh having a filament diameter of preferably 0.5 mm-1 mm, and more preferably about 0.15 mm, and the absorbable or biodegradable central portion is formed as a sagittal section of the hybrid surgical graft from a biodegradable prosthetic material, such as polydioxanone (PDO). The PDO fiber that is used to create the absorbable or bio-degradable mesh may have a filament diameter of between about 0.1 mm to about 4 mm. The preferred diameter of the PDO fiber will depend on the desired resorption time. Those skilled in the art will recognize that the larger the diameter of the PDO fiber, the longer the PDO mesh will remain in the body. A person having ordinary skill in the art will likewise be able to determine the appropriate mesh thickness for each patient depending upon the patient's specific condition.

The weave pattern or other configuration for the meshes of a hybrid surgical graft formed as disclosed herein may be configured in any manner known to those of ordinary skill in the art. In one particular configuration, a warp knitting configuration may be used.

In one configuration of a hybrid surgical graft formed as disclosed herein, PDO #5-0 USP Suture Material may be used to form the central biodegradable portion, and PP-polypropylene monofilament with a diameter of 0.15 mm may be used to form the non-absorbable portion lateral and bridge portions of the hybrid surgical graft. Each material may be warp knitted. The differing materials are preferably attached by wale stitches made of PDO.

Methods of attaching the grafts described herein are consistent with those commonly practiced in the art. It will be understood by those of ordinary skill in the art some of the steps described below may in certain instances be omitted while still allowing for proper installation of a graft for POP repair. Those of ordinary skill in the art will also recognize that the identified steps may be conducted in a different order without impacting the effectiveness of the graft.

In one such exemplary method of using a hybrid surgical graft formed as disclosed herein, the tail of the graft is first cut to fit the patient's vagina. The fitted hybrid surgical graft is then inserted into the patient's vagina and set into place beneath the prolapsed area. In certain instances, sutures may be used to attach the one or more arms to the pelvic sidewall, muscles, or ligaments. The arms are then adjusted to set the graft in place. Fixation means other than sutures may be suitable and used in various situations as will occur to those of ordinary skill in the art.

Experimental Results

The inventor has commissioned the performance of the following studies validating the devices and methods disclosed in the instant application: (1) UMBC Hybrid Mesh PDO/PP First Generation 03012014-Fabric Material Characterization Testing; (2) Development of a Novel Hybrid Mesh for Treatment of Pelvic Organ Prolapse; and (3) Histological appearance of rabbit vaginal and mesouterine tissue following implantation of meshes in a model of cystocele and rectocele. The following discussion briefly presents the above studies and corresponding results.

Report 1: UMBC Hybrid Mesh PDO/PP First Generation 03012014-Fabric Material Characterization Testing (performed by Mark A. Sunderland, Textile Engineer).

Purpose: Gather data on fabric characterization of UMBC Hybrid Mesh PDO/PP First Generation 03012014 Material Characterization. This testing will provide guidance for the preparation of a pre-market material characterization for an implantable surgical UMBC Hybrid Mesh PDO/PP First Generation 03082014 fabric form incorporating certain aspects of an embodiment of the invention. The data to be collected will include the following; pore-size, mesh density, thickness, stitch pattern (cpi and wpi), stiffness, and ball burst strength and grab break.

Scope: This test protocol applies to UMBC Hybrid Mesh PDO/PP First Generation 03012014-Fabric.

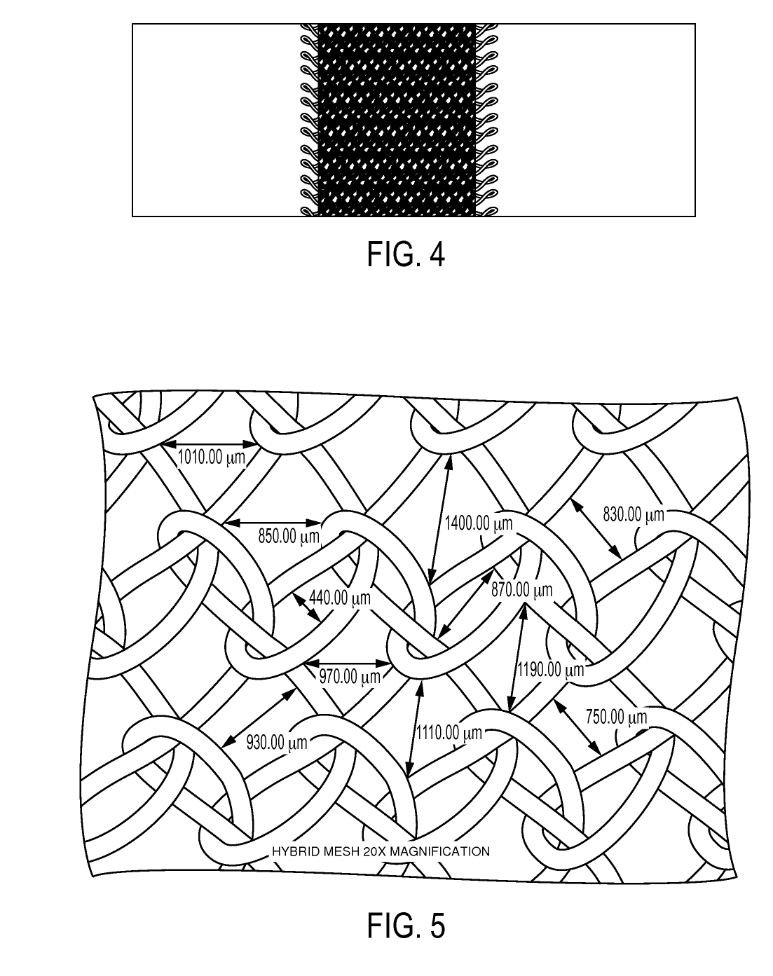

Fabrication Details:
Specimen: UMBC Hybrid Mesh PDO/PP First Generation 03012014-Fabric.
Warp Knit Construction
Dimensions of Sample (width) Left to right: 20 mm-PP; 15 mm-PDO (with transitional area); 20 mm-PP.
Yarn/Fiber: PDO #5-0 USP Suture Material (Blue); PP-polypropylene monofilament diameter 0.006″ (0.15 mm)
Knitting: PDO lateral section consist of 7 wale stitches in width made up of PDO. 1 wale stitch on either side of 7 PDO wale stitches is described as the transitional zone. Transitional zone is alternating PDO and PP stitches. Adding all area in the PDO lateral section total is 9 wales in width.
   Images of the prepared samples are shown in FIGS. 4-5.

Report 2: Development of a Novel Hybrid Mesh for Treatment of Pelvic Organ Prolapse (study performed by Synechion, Inc.—study no. SYN1401MZV).

Introduction: Macroporous non-absorbable monofilament meshes commonly used for the treatment of pelvic organ prolapse are often associated with problems related to their exposure or erosion into the vagina. A mesh has been designed in accordance with certain aspects of an embodiment of the invention to avoid these problems by incorporating a central portion composed of absorbable mesh. A prototype hybrid mesh sheet is provided consisting of two parallel panels of non-absorbable filament material separated by a central panel of absorbable filament (FIG. 4).

Purpose: To evaluate the formation of peri-vaginal adhesions after the placement of a hybrid mesh in accordance with certain aspects of an embodiment of the invention in a rabbit model.

Method: After preparation for sterile surgery, and induction of anesthesia using isoflurane, 18 female New Zealand white rabbits weighing 2.5-3.02 kg underwent laparotomy. Following a midline incision and delivery of the uterus into the wound, animals were assigned to receive one of two kinds of meshes: A) Prolene Soft Polypropylene Mesh (Ethicon, Somerville, N.J.), or B) a novel Hybrid mesh composed of polypropylene with a central portion of polydioxanone. Meshes were sutured in place to the uterine ligament over the vagina on both its anterior and posterior surfaces.

Three animals from each group were evaluated at approximately 6, 13 and 26 weeks after surgery. The extent and severity of adhesions was noted as well as the degree of inflammation assessed grossly. Adhesions were assessed both in the central zone overlaying the vagina and the “left” and “right” zones which did not overlay the vagina.

Results: At six weeks, meshes were firmly adherent to the vagina and uterine ligament with ingrowth of connective tissue between the mesh interstices for both kinds of meshes. Adhesions typically formed to the bladder on the anterior side. On the posterior side, adhesions formed to a small extent, from the mesh to the part of the rectum in, or close to the cul-de-sac. A trend was observed of slightly more extensive adhesion formation in animals treated with the hybrid mesh, compared to those treated with the PROLENE mesh. This trend was seen both for the anterior and posterior sides, as well as the center or right and left zones. For the anterior side, in the left and right zones combined, adhesions occurred over 62.5+14.2% of the mesh area for the Prolene mesh and 75.8+12.1% for the hybrid mesh. The corresponding figures for the central anterior zone were 78.3+21.7% and 90+5.8% respectively. For the posterior side, in the left and right zones combined, adhesions occurred over 1.3+0.7% of the mesh area for the Prolene mesh and 6.7+6.7% for the hybrid mesh. The corresponding figures for the central anterior zone were 5.0+0% and 7.3+6.3% respectively. The opposite trend was noted for the tenacity of adhesions or the degree of inflammation near the implantation site in all zones.

At 13 weeks, slightly more extensive adhesion formation was seen in animals treated with the hybrid mesh, compared to those treated with the PROLENE mesh. This trend was seen both for the anterior and posterior sides, as well as the center or right and left zones. For the anterior side, in the left and right zones combined, adhesions occurred over 40.8+11.7% of the mesh area for the Prolene mesh and 40+11.6% for the hybrid mesh. The corresponding figures for the central anterior zone were 25+18% and 71.7+23.5% respectively. For the posterior side, in the left and right zones combined, adhesions occurred over 6.7+1.7% of the mesh area for the Prolene mesh and 26.7+13.1% for the hybrid mesh. The corresponding figures for the central anterior zone were 5.0+2.9% and 10+7.6% respectively. No clear trends could be discerned regarding the tenacity of adhesions. No evidence of inflammation was evident by gross inspection.

At 26 weeks the pattern of adhesion formation in animals treated with Prolene mesh was similar to that seen at earlier time points. In one animal treated with hybrid mesh, adhesions were absent in the central portion corresponding to the degradable section of mesh. In the remaining two animals in this group, a membrane was found covering the vagina on the anterior side. This was part of an adhesion that included the bladder. This was easily removed with no adhesions to the vagina. Adhesions were typically found on the lateral sides in animals treated with the hybrid mesh.

The non-degradable portions of the hybrid mesh were typically ingrown with fibrous tissue and attached to the underlying structures. This was true for much of the Prolene mesh, although on occasions the mesh was loosely adherent to the underlying structure. There was no evidence of bowel, bladder or vagina erosion at 26 weeks in any animal.

The trend observed at 6 and 13 weeks of slightly more extensive adhesion formation in animals treated with the hybrid mesh, compared to those treated with the PROLENE mesh, was reversed at 26 weeks. For the anterior side, in the left and right zones combined, adhesions occurred over 56.7+3.3% of the mesh area for the Prolene mesh and 48.3+4.2% for the hybrid mesh. The corresponding figures for the central anterior zone were 53.3+17.6% and 43.3+29.6% respectively. For the posterior side, in the left and right zones combined, adhesions occurred over 31.7+15.9% of the mesh area for the Prolene mesh and 25.0+10.1% for the hybrid mesh. The corresponding figures for the central anterior zone were 28.7+25.7% and 1.7+1.7% respectively.

Discussion: This interim report describes the feasibility of producing adhesions between a mesh placed around the vagina for the anterior side, mainly to the bladder, as occurs in clinical practice.

The placement of both types of mesh was easily accomplished. The hybrid mesh tended to curl along the two edges perpendicular to the direction of the central stripe. Adhesion formation on the posterior side to the bowel and other structures was disappointing. Both meshes incorporate well into the wall of the vagina at early time points.

At early time points, there was a slightly increased tendency towards adhesion formation for the hybrid mesh compared with the Prolene Mesh in the left, right and central zones, and both anteriorly and posteriorly. This trend was reversed at 26 weeks.

With time the trend of adhesion formation varied greatly by location and type of mesh. On the lateral anterior margins, no trend could be discerned for Prolene mesh, but adhesions for the hybrid mesh were reduced. In the central zone, both Prolene and hybrid mesh adhesions were reduced. For the posterior side adhesion formation increased in both zones for Prolene mesh treated animals, and were slightly increased laterally for the hybrid mesh group. Centrally in this latter group adhesions were down, against a background of low adhesion formation. The small sample sizes make further interpretation difficult.

Most significantly, once the central portion of the hybrid mesh had degraded at 26 weeks, adhesions were absent to the anterior vagina. In two animals a membrane had formed that was part of a bladder adhesion. Whether this membrane would be strong enough to prevent bladder prolapse requires further study.

Conclusion: The model was capable of forming peri-vaginal adhesions on the anterior side to a standard polypropylene mesh. Adhesions to the posterior side were usually sparse. At 6 and 13 weeks, the extent of adhesions in Prolene mesh animals was generally slightly lower than in hybrid mesh-treated animals. This trend was reversed at 26 weeks. Significantly, once the central portion of the hybrid mesh had degraded at 26 weeks, adhesions were absent to the anterior vagina. In two animals a membrane had formed that was part of a bladder adhesion. A determination of whether this membrane would be strong enough to prevent bladder prolapse would be informed by further study.

Test Article: Hybrid meshes were produced by Mark A. Sunderland, Manager Academic Operations, Textile Engineer and Strategist Kanbar College-Design Engineering and Commerce, Philadelphia University 4201 Henry Avenue, Philadelphia, Pa. 19144. The meshes were supplied as individually packed and sterilized sheets approximately 9×5.5 cm. Running the length of the mesh was the central strip of knitted, dyed polydioxanone 1.5 cm in width. Knitted into this strip on either side were strips of polypropylene, about 2 cm wide. Meshes were sterilized by Ethylene Oxide by Isomedix, STERIS Isomedicx Services, 3459 South Clinton Avenue, NJ 07080.

Reference Articles: PROLENE Soft Polypropylene Mesh (Non-absorbable synthetic surgical mesh) was obtained from Ethicon, Inc., (Somerville, N.J.) as a 10×10″ mesh. Product Code SPLI, Lot DAB333, expiry date January 2016. The mesh consisted of alternating stripes of undyed (about 1.7 cm) and blue-dyed (about 4 mm) polypropylene. Group Assignments: There were two groups, each of 9 animals. Group A (Control) treatment performed with Prolene Mesh. Group B (Test) treatment performed with novel hybrid mesh. Three animals from each group were evaluated at approximately 42, 91 and 182 days.

This report describes the feasibility of producing adhesions between a mesh placed around the vagina for the anterior side, mainly to the bladder, as occurs in clinical practice. Adhesion formation on the posterior side to the bowel and other structures was disappointing.

The placement of both types of mesh was easily accomplished. The hybrid mesh tended to curl along the two edges perpendicular to the direction of the central stripe.

Both meshes incorporate well into the wall of the vagina. At early time points, there is a slightly increased tendency towards adhesion formation for the hybrid mesh compared with the Prolene Mesh in the left, right and central zones, and both anteriorly and posteriorly. The opposite trend was noted for the tenacity of adhesions or the degree of inflammation near the implantation site in all zones. This trend was reversed at 26 weeks.

With time the trend of adhesion formation varied greatly by location and type of mesh. On the lateral anterior margins, no trend could be discerned for Prolene mesh, but adhesions for the hybrid mesh were reduced. In the central zone, both Prolene and hybrid mesh adhesions were reduced.

For the posterior side adhesion formation increased in both zones for Prolene mesh treated animals, and were slightly increased laterally for the hybrid mesh group. Centrally in this latter group adhesions were down, against a background of low adhesion formation. The small sample sizes make further interpretation difficult.

Most significantly once the central portion of the hybrid mesh had degraded at 26 weeks, adhesions were absent to the anterior vagina. In two animals a membrane had formed that was part of a bladder adhesion. A determination of whether this membrane would be strong enough to prevent bladder prolapse would be informed by further study.

Report 3: Histological appearance of rabbit vaginal and mesouterine tissue following implantation of meshes in a model of cystocele and rectocele (study performed by Synechion, Inc.—study no: SYN1401ZSX).

Introduction: Macroporous non-absorbable monofilament meshes commonly used for the treatment of pelvic organ prolapse are often associated with problems related to their exposure or erosion into the vagina. A mesh has been provided in accordance with certain aspects of an embodiment of the invention to avoid these problems by incorporating a central portion composed of absorbable mesh. In a previous study (SYN1401MZV) the gross effects of this novel mesh were evaluated in a rabbit model. Tissue specimens were collected in that study but not analyzed.

Purpose: To describe the histological appearance of rabbit vaginal and mesouterine tissue following implantation of meshes in a model of cystocele and rectocele, using tissue specimens generated in study SYN1401MZV.

Method: Tissue specimens were generated in study SYN1401MZV after surgical fixation in rabbits of one of two kinds of mesh to the uterine ligament over the vagina on both its anterior and posterior surfaces. The two meshes were A) Prolene Soft Polypropylene Mesh (Ethicon, Somerville, N.J.), or B) a novel hybrid mesh composed of polypropylene with a central portion of polydioxanone (as shown in FIG. 8). Meshes were sutured in place. Three animals from each group were evaluated at approximately 6, 13 and 26 weeks after surgery. Upon harvest the anterior surface was marked using a suture for orientation purposes. Tissues were fixed in neutral buffered formalin and an approximately 15×40 mm rectangle trimmed to include a portion of the vagina and right uterine ligament. Dye was used to identify the anterior and posterior surfaces after sectioning. Tissues were processed using standard techniques. Two sets of three serial sections were taken approximately 5 mm from the edge of the trimmed tissue. In each set, one section was stained with H&E, one with trichrome and one section was left unstained. The sections were examined by a veterinary pathologist and evaluated for evidence of tissue reaction, fibrosis, material degradation and foreign body reaction.

Results: Meshes, regardless of type became encapsulated by fibrous tissue associated with inflammatory responses typical of degradable (PDS) or non-degradable and relatively inert (polypropylene) materials. There was no evidence of degeneration of the polypropylene mesh in any of the animals.

The patterns of inflammatory changes shown for polypropylene whether in the lateral region of the hybrid mesh, or in the lateral and central regions of the Prolene mesh, are essentially similar. Scores remained mild to moderate until 13 weeks and increased a little at 26 weeks. For the hybrid mesh, the inflammatory reaction to the polypropylene portion remained low. Inflammation for the PDS in the central portion of the hybrid mesh shows a different pattern with an initially stronger reaction, attenuating with time.

The progression of fibrosis with time is similar for polypropylene regardless of the type of mesh, generally increasing with time. However, for the PDS in the central portion of the hybrid mesh, fibrosis peaks at 12 weeks and starts to attenuate, presumably as the material degrades.

By 26 weeks PDS had degraded and its presence indicated by clusters of inflammatory cells containing digested remnants of the filaments.

Discussion & Conclusion: Meshes are used to treat rectocele or cystocele prolapses. It is possible that a fibrosis elicited by a mesh such as the PDS portion of the hybrid mesh, if sustained, may provide sufficient support to prevent recurrent prolapse. Alternatively, the fibrosis that remains on the lateral wings of the hybrid mesh may undergo contracture significant enough to provide support for the vagina, thereby reducing the opportunity for relapse of prolapse. Both hypotheses would likely benefit from further investigation.

In the foregoing description, the invention has been described with reference to specific embodiments thereof. It will, however, be evident to those of ordinary skill in the art that various modifications and changes may be made thereto without departing from the broader spirit and scope of the invention. The specification and drawings are, accordingly, to be regarded in an illustrative rather than a restrictive sense. Throughout this specification and the claims, unless the context requires otherwise, the word “comprise” and its variations, such as “comprises” and “comprising,” will be understood to imply the inclusion of a stated item, element or step or group of items, elements or steps. Furthermore, the indefinite article “a” or “an” is meant to indicate one or more of the item, element, or step modified by such article. Having now fully set forth the preferred embodiments and certain modifications of the concept underlying the present invention, various other embodiments as well as certain variations and modifications of the embodiments herein shown and described will obviously occur to those skilled in the art upon becoming familiar with said underlying concept. It should be understood, therefore, that the invention may be practiced otherwise than as specifically set forth herein.

What is claimed is:

1. An implantable graft comprising:
a support structure configured for attachment to a patient's tissue formed of a non-absorbable mesh; and
a biodegradable portion positioned at an interior portion of said non-absorbable mesh.

2. The implantable graft of claim 1, said biodegradable portion having long side edges extending parallel to a major axis of the graft, and short side edges extending perpendicular to the major axis of the graft.

3. The implantable graft of claim 2, wherein said biodegradable portion is attached along a first one of said long side edges to a first section of said non-degradable mesh, and is attached along a second one of said long side edges to a second section of said non-degradable mesh.

4. The implantable graft of claim 3, wherein said short side edges of said biodegradable portion form a portion of an outer perimeter of said graft, and wherein said graft is configured to comprise two separated support structure sections following dissolution of said biodegradable portion.

5. The implantable graft of claim 3, said non-degradable mesh further comprising an anterior bridge of non-absorbable mesh adjacent a first short side edge of said biodegradable portion and forming a first portion of an outer perimeter of said graft, and a posterior bridge of non-absorbable mesh adjacent a second short side edge of said biodegradable portion and forming a second portion of an outer perimeter of said graft, and wherein said non-degradable mesh is configured to form an annular ring following dissolution of said biodegradable portion.

6. The implantable graft of claim 5, wherein each of said anterior bridge of non-absorbable mesh and said posterior bridge of non-absorbable mesh has a width of up to 2 mm.

7. The implantable graft of claim 1, wherein said biodegradable portion is formed of polydioxanone filament having a filament diameter of 0.1 mm to 4 mm.

8. The implantable graft of claim 7, wherein said non-absorbable mesh is formed of polypropylene mesh having a filament diameter of 0.5 mm to 1 mm.

9. A mesh for use in an implantable graft comprising:
a biodegradable portion; and
a non-absorbable mesh attached to at least a portion of an outer perimeter edge of said biodegradable portion.

10. The mesh of claim 9, wherein said non-absorbable mesh forms a support structure of a surgical graft that is configured for attachment to a patient's tissue.

11. The mesh of claim 10, said biodegradable portion having long side edges extending parallel to a major axis of the graft, and short side edges extending perpendicular to the major axis of the graft.

12. The mesh of claim 11, wherein said biodegradable portion is attached along a first one of said long side edges to a first section of said non-absorbable mesh, and is attached along a second one of said long side edges to a second section of said non-absorbable mesh.

13. The mesh of claim 12, wherein said short side edges of said biodegradable portion form a portion of an outer perimeter of said graft, and wherein said graft is configured to comprise two separated support structure sections following dissolution of said biodegradable portion.

14. The mesh of claim 12, said non-absorbable mesh further comprising an anterior bridge of non-absorbable mesh adjacent a first short side edge of said biodegradable portion and forming a first portion of an outer perimeter of said graft, and a posterior bridge of non-absorbable mesh adjacent a second short side edge of said biodegradable portion and forming a second portion of an outer perimeter of said graft, and wherein said non-absorbable mesh is configured to form an annular ring following dissolution of said biodegradable portion.

15. The mesh of claim 14, wherein each of said anterior bridge of non-absorbable mesh and said posterior bridge of non-absorbable mesh has a width of up to 2 mm.

16. The mesh of claim 9, wherein said biodegradable portion is formed of polydioxanone filament having a filament diameter of 0.1 mm to 4 mm.

17. The mesh of claim 16, wherein said non-absorbable portion is formed of polypropylene mesh having a filament diameter of 0.5 mm to 1 mm
